(19) United States
(12) Patent Application Publication
(10) Pub. No.: US 2007/0179373 A1
(43) Pub. Date: Aug. 2, 2007

(54) Integrated patch and assay device with visual detection means

(75) Inventor: Allan D. Pronovost, San Diego, CA (US)

Correspondence Address: FOLEY & LARDNER LLP, 111 HUNTINGTON AVENUE, 26TH FLOOR, BOSTON, MA, 02199-7610, US

(73) Assignee: PROVEX TECHNOLOGIES, LLC.

(21) Appl. No.: 11/644,496

(22) Filed: Dec. 22, 2006

Related U.S. Application Data
(60) Provisional application No. 60/753,213, filed on Dec. 22, 2005.

(51) Int. Cl. A61B 5/00; G01N 21/00; G01N 31/22

(52) U.S. Cl. 600/362; 422/56

(57) ABSTRACT

Methods and apparatus for collecting a fluid sample using an integrated collection device are disclosed. The integrated collection device includes an analyte detector and a gradient means to actively and rapidly drive the transport of a sample fluid from the point of contact to the point of detection and reading. The analyte detector can include a visually-read colorimetric detector using a chemical or enzymatic detection process. The gradient means can include physical and/or chemical processes as described in more detail herein. In some embodiments, the device is provided as an occlusive patch. Preferably, the test device provides a reading immediately upon fluid contact with the analyte detector. Consequently, the time-to-result is sample volume and transport time dependent. By providing immediate or nearly immediate reading of results, the device is particularly useful in those applications in which immediate results are advantageous.

### CROSS-REFERENCE TO RELATED PATENT APPLICATIONS

This application claims priority from U.S. Provisional Application Ser. No. 60/753,213, filed on Dec. 22, 2005, incorporated herein by reference in its entirety.

### FIELD OF THE INVENTION

The present invention relates generally to assay devices and more particularly to assay devices combined with a fluid sampling capability.

### BACKGROUND OF THE INVENTION

The use of non-occlusive patch technology for the analysis of alcohol and drugs of abuse is under development. Limitations associated with such technologies include excessively long wear times, patch leakage, back diffusion problems, lack of analyte binding to the pad layer to prevent resorption by the skin. Other limitations include the need for normalization and correction of analyte concentration for actual sweat volume over time (over long term wear) through a supplemental procedure, such as the detection of potassium values by flame photometry in a laboratory. Accordingly, such devices are not well suited for applications in which a rapid sample and analyte detection process is necessary.

### SUMMARY OF THE INVENTION

What is needed are devices and methods capable of collecting a sample during a short interval combined with an rapid analyte detection process.

Various embodiments of the present invention can provide an integrated sample collection device incorporating an analyte detector, and a gradient means to actively and rapidly drive the transport of sample fluid from the point of contact to the point of detection and reading.

In one aspect, the invention relates to an integral fluid collection device including at least three internal layers. A first layer includes a hydrophilic nanofiltration material adhered to a second layer. The second layer includes absorbent material adhered to a third layer. The third layer includes an assay detection zone having integrated direct chemical calorimetric or enzymatic-based colorimetric detection suitable for detecting an analyte. The third layer is adhered to a fourth layer that includes a visually-clear occlusive material. The fourth layer is external to the first, second and third layers, having an area greater than the at least three internal layers so as to overlap the three internal layers.

In another aspect, the invention relates to a process for detecting a target analyte obtained from a sample. The process includes obtaining a patch including at least three internal layers. The first layer includes a hydrophilic nanofiltration material adhered to a second layer. The second layer includes an absorbent material adhered to a third layer. The third layer includes an assay detection zone having integrated direct chemical colorimetric or enzymatic-based colorimetric detection suitable for detecting an analyte. The third layer is adhered to a fourth layer, which has a visually-clear occlusive region. The fourth layer is external to the first, second, and third layers and has an area greater than the three internal layers so as to overlap the three internal layers applying the patch to the sample surface; and viewing through the visually-clear occlusive region a colorimetric change in the patch, wherein the colorimetric change indicates the presence of the target analyte.

In yet another aspect, the invention relates to a process for producing an analyte detection patch. The process includes providing a first layer having hydrophilic nanofiltration material. A second layer is provided having absorbent material. A third layer is provided comprising an assay detection capability including integrated direct chemical colorimetric or enzymatic-based calorimetric detection chemistry suitable for detecting an analyte and a fourth layer is provided comprising a visually-clear occlusive material. The fourth layer is external to the first, second and third layers and has an area greater than these three internal layers. In some embodiments, the fourth layer also having a release liner and an adhesive zone peripheral to the three internal layers.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is a schematic diagram providing a cross-sectional view of an embodiment of a multi-layer patch in accordance with the principles of the invention.

FIG. 1B is a schematic diagram providing a cross-sectional view of another embodiment of a multi-layer patch in accordance with the principles of the invention.

FIG. 2 is a schematic diagram providing a cross-sectional view of yet another embodiment of a multi-layer patch disposed on a fluid vessel, shown in partial cut-away, in accordance with the principles of the invention.

FIG. 3A is a schematic diagram providing a planar view of an embodiment of a multi-layer circular patch in accordance with the principles of the invention.

FIG. 3B is a schematic diagram providing a cross-sectional view of the circular patch of FIG. 3A.

FIG. 4A is a schematic diagram providing a top view of an embodiment of a multi-layer patch attached to an outer surface of fluid vessel, shown in partial cut-away, in accordance with the principles of the invention.

FIG. 4B is a schematic diagram providing a cross-sectional view of the multi-layer patch of FIG. 4A.

FIG. 5 is a schematic diagram providing a planar view of another embodiment of a multi-layer patch attached to an outer surface of a filled fluid vessel, shown in partial cut-away, in accordance with the principles of the invention.

### DETAILED DESCRIPTION OF THE PREFERRED EMBODIMENTS

A description of preferred embodiments of the invention follows.

The analyte detector can include a visually-read calorimetric detector using a chemical or enzymatic detection process. The gradient means can include physical and/or chemical processes as described in more detail herein. In some embodiments, the device is provided as an occlusive patch. Preferably, the test device provides a reading immediately upon fluid contact with the analyte detector. Consequently, the time-to-result is sample volume and transport time dependent. By providing immediate or nearly immediate reading of results, the device is particularly useful in those applications in which immediate results are advantageous.

At least one example of an application that would benefit from such immediate reading or results is dispensed beverage screening. The integrated visual patch assay device can be used for immediate sugar detection. The device can be mounted on the outside of a vessel containing a dispensed soft drink to provide a visual indication to consumers, notifying them of the sugar or sugar-free status of a beverage. Alternatively or in addition, the device can be used in a similar manner, adapted to detect other analytes, such as caffeine in coffee beverages to indicate and assure consumers of caffeine-free status of a beverage. Another application includes a rapid point-of-care visual screening for impaired tolerance (pre-diabetes) or diabetes in consumers using an integrated patch.

In some embodiments, the device is configured as a single-use, patch sample-collection-device, with integrated, visually-read colorimetric detection means for the in situ determination of analyte(s) in a transported fluid. The technology incorporates a physical and/or chemical gradient means to actively drive the transport of fluid from a sample collection site to the analyte detector. In some embodiments, a patch sample collection device includes an integrated colorimetric chemical or enzymatic assay detection means within the patch providing visual colorimetric determination of an analyte. Such an integrated patch and assay device when used in combination with a fluid container has particular application to sugar or caffeine detection in dispensed beverage fluids such as soft drinks. Such an integrated patch and assay device can also be adapted to contact an animal body surface, for example, to detect carbohydrates in mammalian dermal fluid. Such a detection capability can serve as a screening basis for impaired tolerance or diabetes, and other applications.

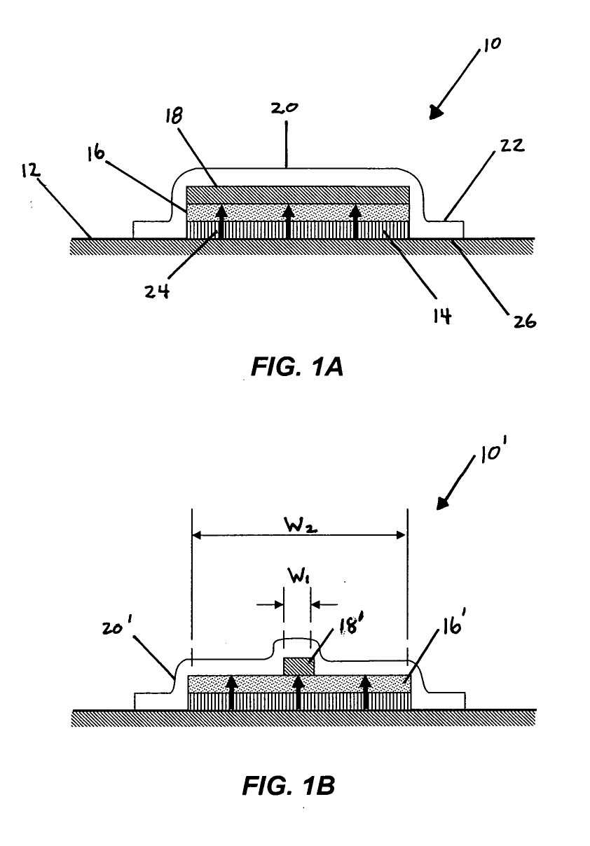

In some embodiments, the device includes a transparent, multi-layered, occlusive, adhesive patch that will not transpire or allow fluid from within to leak out. A cross-section of an exemplary multi-layered device 10 is shown in FIG. 1. The device 10 includes a first layer 14 having a bottom surface adapted for placement against a surface to be sampled 12. A second layer 16 is placed in communication with a top surface of the first layer 14. A third layer 18 is placed in communication with at least a portion of a top surface of the second layer 16, the three layers forming a stack. An over-layer 20 covers the entire stack and includes a perimeter portion 22 extending outward and away from the stack. Preferably, the over-layer 20 is substantial impermeable to water, resulting in a occlusive device.

The occlusive design serves several purposes. First, the occlusive design effectively drives fluid transport allowing the patch layers 14, 16, 18 to saturate with fluid rapidly. Alternatively or in addition, the occlusive design facilitates retention of a majority of the transported fluid. The over-layer 20 operates to physically contain any transported fluid to assure an adequate volume remains to be processed by a chemical or enzymatic detection means. The occlusive design also promotes yielding results upon a first pass, without a prolonged assay time, or patch wear time.

The occlusive design also provides a leak-proof barrier around the outside of the patch at the point of application to facilitate fluid transport from a sample surface to the device 10. When used with fluid vessels, such as beverage containers, the occlusive design prevents leakage of any fluid contents dispensed therein. The occlusive design also prevents back transport of chemicals contained on patch layers distal from the application site by minimizing contact, wear time, and time-to-result. The occlusive adhesive patch 10 can be integrated onto the external surface of dispensed beverage containers by manufacturing beverage to include such a patch device, by using any of the designs exemplified herein.

The patch 10 incorporates the use of either a multiple zone lateral flow or multilayered flow-through stack 24 of selected media, as membranes or web material with or without pre-conditioning or treatment of each layer. As shown in FIG. 1, the layers of media 14, 16, 18 can be positioned laterally along a sample surface. In some embodiments, one or more of the layers 14, 16, 18, 20 can be adhered together with an adhesive. Preferably, the adhesive is an aqueous-permeable, porous, double-stick adhesive to minimize interference with fluid transfer within the device 10. The zones or stack layers 14, 16, 18 are appropriately arranged (in some cases centered) under a transparent, non-water permeable outer polymer film layer 20. This layer can include polyethylene terephthalate, such as MYLAR, a registered trademark of Du Pont De Nemours and Company. Viewing and reading of the assay results can be accomplished through the transparent layer 20 read zone at a specified assay read time.

The perimeter 22 of the outer film layer 20 that comes in contact with the surface area of application 12, can include a peripheral adhesive zone. Preferably the adhesive is a non-aqueous permeable double-stick adhesive to promote containment of fluid within the device. This outer adhesive layer surrounds the lateral flow or multilayered flow-through stack. One side of outer layer's peripheral adhesive can be pre-attached to the outer MYLAR film layer at least along the perimeter 22 to provide a barrier. When using a pre-attached adhesive in this manner, the side to be adhered to the skin 26 requires the removal of a protective release liner before application to a sample surface 12.

Beverage container patches can be pre-adhered to the outside wall of a fluid vessel in communication with a fluid access means as described herein. For dermal applications, the entire patch is preferably pre-packaged in a sealable envelope, or pouch and appropriately labeled with instructions for wear and use.

In some forms, the device includes a multilayered stack having four layers adhered together with double stick adhesive. It is understood by those skilled in the art that the number of actual layers depends upon the application, wherein there may be fewer or more than the exemplary four layers described herein.

Continuing with the exemplary four-layered device 10 of FIG. 1, the first central layer 14 closest to the sample surface 12 (e.g., fluid vessel or skin) can include an ultra-thin hydrophilic, plastic nanofiltration material. The first layer 14 is adhered to the second layer 16 with an aqueous permeable adhesive at one or more contact points. The second central layer 16 can include cellulosic or a cellulosic/glass fiber blend of absorbent material. The second layer 16 is similarly adhered to the third layer 18 with an aqueous permeable adhesive at one or more contact points. The third layer 18 includes an assay detection layer or zone of a preferred size. The assay detection layer 18 includes integrated color or enzyme chemistry providing colorimetric detection of target analytes. The third layer 18 can be adhered to the fourth layer 20 again using an aqueous permeable adhesive at one or more contact points. The fourth external or over-layer 20 includes an occlusive polymer film, such as MYLAR, that includes at least one region that is a visually-clear to provide colorimetric readout capability. The fourth layer 20 can have an area greater than the area of the stack of the prior three layers 14, 16, 18 and as such is designed to overlap the three central layers 14, 16, 18. The overlap portion 22 of the fourth layer 20 also contains a peripheral aqueous permeable adhesive zone and release liner to serve as the means for adherence to the sample, or application area 12.

The first central layer 14 is the layer that comes in intimate contact with the application area 12 and has an active role in contact and transport. Preferably, the first layer 14 also provides a barrier from the retained chemistry of the third layer 18. Typically, the application area 12 is dry until application of the device 10, whereafter fluid transport is initiated. In the case of beverage testing, fluid transport can be initiated by simply filling a dispensed beverage into a suitably modified fluid vessel. In the case of a dermal patch, fluid transport can be initiated by simply applying the device to the skin. Preferably, the first layer 14 has one or more properties that promote its desired functionality. A first property includes a high hydrophilicity to allow for ready fluid contact and transport. A second property is superior adhesion under wet conditions, essentially behaving like a skin. Superior adhesion under wet conditions necessitates that the material be ultra-thin and pliable to allow for ready adhesion to both the wet surface and the second layer. A third property is that the first layer 14, or membrane be chemically inert at least in that it does not retain analyte or the fluid necessary to conduct the assay. Such a property is provided by certain polycarbonate plastic, alumina oxide metallic, or ceramic membranes such.

Concentration of analyte through moisture removal is thus avoided to minimize variation of transported fluid concentration of analyte from the true concentration of analyte in the fluid being tested prior to transport. Yet another property is that the membrane 14 have discrete, uniform diameter, cylindrical, straight-through pores (as in bullet-like holes) that act as a molecular sieve to allow for immediate passage of fluid. The density of such pores should be extremely high to allow for ready fluid flow at a high transport rate. Preferably the pores have a small diameter (i.e., nano-pores) to support a high density of the pores allowing a hydrophilic fluid flow. The pore size (diameter) of these membranes can be extremely small and uniform to facilitate the molecular nanofiltration of sample. Nanofiltration of sample is preferred to prevent transport of interfering debris to the assay means in the third layer 18, and to prevent back transport of assay biomaterials, e.g. enzyme, to the area of application 12. Most conventional membranes do not have discrete uniform cylindrical pores in the nanometer range and are considerably larger (1000×) in size cutoff with a non-discrete pore size (these are known as micron filters). In addition, conventional cast membranes with overlapping fibers are not suitable for this application due to the lack of discrete cylindrical pores.

Hydrophilic inert nanopore membranes are particularly well suited for use in the first layer 14. Such nanopore membranes are available having pore size less than about 200 nm (e.g., a range from about 2 nm to about 200 nm). Preferably there are more than 1×10⁸ pores/cm² (e.g., a range from about 1×10⁷ pores/cm² to about 1×10⁹ pores/cm²). Preferably the membrane has a nominal thickness of less than about 10 micrometer (with a range from about 5 micrometers to about 20 micrometers), a bubble point greater than about 50 pounds per square inch (PSI), and a water flow rate range between about 0.1 ml/minute/cm² to about 20 ml/minute/cm² depending on pore size. Examples include ion track-etched polycarbonate membranes (NUCLEPORE, a registered trademark of Nuclepore Corp., Pleasanton Calif., and commercially available from Osmonics, Minnetonka, of Minn., or SPI Supplies, of Westchester, Pa.), Anopore Inorganic Aluminum Oxide Membranes (also commercially available from SPI Supplies), and/or Steriltech ceramic membranes (commercially available from Steriltech Corporation, of Kent, Wash.), and/or any custom nanofabricated, uniform morphology, self-organized, anodic alumina or ceramic nanodevice array constructed for thin film separation purposes.

The recent advent of these nanomembranes provides a preferred technical means for rapidly and selectively removing insoluble or soluble materials from samples in the molecular size range of 2 nm to several hundred nm in a rapid fashion (i.e., less than about 30 seconds). In contrast, conventional approaches used for nano-range separations are generally slow and tedious. For example, ultracentrifugation of a sample provides a comparable separation, but takes up to 12 hrs or more at 100,000×g in an ultracentrifuge. Nanofiltration of a fluid sample with a 2 nm pore size nanofilter would leave it in a state wherein filtrate only contains soluble material below approximately 1,500 Daltons and for a 20 nm pore size nanofilter, approximately 15,000 Daltons. And as such, discrete uniform pores do not allow the transport of globular macromolecules of higher molecular weight in either direction.

In addition the use of thin filmed, ion-track etched polycarbonate nanofilter membranes provides good skin contact under wet conditions owing to the inherent ultra-thin pliable nature of the polycarbonate membrane.

The second central layer 16 can include an absorbent material, such as cellulosic, cellulosic/glass fiber blend, or other suitable absorbent material in contact with the first and third layers 14, 18 through aqueous permeable adhesive or other suitable means (such as pressure adhesion, ultrasonic welding, thermal adhesion, etc.). The second layer 16 serves at least two important purposes. A first purpose is to help attract or “pull” the water being transported into itself 16 and subsequent layers 18. That can be accomplished through composition of the layer 16 and/or through pretreatment of the second layer 16 to induce transport. A second purpose for the second layer 16 is to serve as a reservoir for the transported fluid so as to supply it to the third layer 18, which typically requires a sufficient quantity or concentration of water and analyte to drive the calorimetric chemical or enzymatic reactions.

The second layer 16 may induce transport from the point of contact through the first layer 14 by a series of induction means. In the case of a dermal patch 10, induction of sweat release is also a desired feature to shorten wear time and time-to-result. Induction is ideally unidirectional reducing back transport into the beverage container or resorption by the skin. To facilitate such, induction of fluid transport necessitates the unidirectional attraction, accumulation, and retention of water. Induction should also be efficient in that fluid is transported as fast as possible to allow an early and timely result. One of the means to induce unidirectional transport is to establish a gradient, namely a gradient or variation of some physical property from the area of contact 12 to the detection means (third layer 18). Various induction means include the following types of gradients: a dryness (moisture) gradient; a hydrophilic gradient for polar water molecules; an absorbent polymer gradient; a chemical equilibrium osmosis gradient; a physical gradient; or the like.

One of the simplest gradients is a moisture gradient. In this case the composition of the material for layer two 16 is selected to absorb water. Cellulose, cellulose/glass fiber, glass fiber, or other blends of membrane materials common in the art can be used for this purpose. As example, various macroporous chromatographic separation media are commercially available from Whatman Inc. of Florham Park, N.J. Such separation media can be selected empirically for desired dryness (low moisture content) in addition to specific migration rates on either a horizontal or vertical flow basis with application to fluid transport. Selected properties that are useful include speed of flow, wicking speed, separation rate, etc. Specific useful materials include Whatman multi-media composite microfiber membranes such as grades 934-AH, or Multigrade GMF with linear or radial wicking times of 50 sec/7.5 cm at 1 um; or S&S grade GF10, 53, etc.

Another suitable media for establishing a unidirectional dryness gradient is to use sheet-form desiccant material containing dispersed silica dioxide. An example of such materials includes Drikette R Desiccant Papers, commercially available from Multisorb Technologies, Inc., of West Seneca, N.Y. Alternatively or in addition, other absorptive materials such as activated alumina, bentonite clay as powder, natural or synthetic powdered zeolites, or ceramic micropheres can be incorporated into the media as well and are available from a variety of sources and are well known in the art.

A hydrophilic gradient can be established for the second layer 16. Hydrophilic is defined as having a very high affinity for polar water molecules. This is accomplished through the selection of materials based on their chemical nature and composition so as to render them innately hydrophilic or at least absorptive. Interaction of these materials with water results in extensive hydrogen bonding with polar water molecules as the means to drive a hydrophilic gradient. Yet because of the relative weakness of hydrogen bonds as compared to covalent bonding, water molecules are still readily available to drive the chemical reactions of the third layer 18.

As example, a surface of fumed silica dioxide nanoparticles, available as a powder, contains extensive hydroxyl groups at a very high surface density all with an extremely high affinity for polar water molecules. In addition, the small “nano” particle size of fumed silica results in an extremely high surface area for fumed silica, up to 400 m²/gm, which not only assures the very high density of hydroxyl functional groups but a high capacity for water molecule attraction and transport. Fumed silica nanoparticles are dispersed into the second layer 16 by a variety of means including direct powder coating, or precoating of membranes in non-aqueous liquid solutions of non-polar, short chain molecules such as glycerol or through dispersion in an evaporating solvent such as alcohol. Examples of suitable fumed silica material include Cabot PTG, EH5, HS5, M5, M5P, and LM130, LM150 or deGussa Aerosil 150, Aerosil 200, COX84, or COX170. The latter two compounds contain alumina oxide to facilitate thixotropy and hence serve as effective rheology modifiers as well.

An absorbent polymer gradient can be established for the second layer 16 through coating of selected media, such as cellulosic membranes, with a super-absorbent material that can retain more than about 100 times its weight in water. Such materials include starch graft copolymers such as poly (2-propenamide-co-2-propenoic acid) available as the sodium or potassium salt, or starch copolymers such as poly (2-propenamide-co-2-propenoic acid, sodium salt). Powdered Water-lock R Superabsorbent starch polymers, available from GPC, with a typical uptake of >130 up to more than 600 times their weight are added to the second layer 16 to setup an effective unidirectional absorbent polymer gradient (e.g., in the direction of the arrows 24 of FIG. 1). The hydrated polymer containing water serves as an in situ formed hydrogel differentiating it from preformed hydrogels and the storage and adhesion issues of dealing with such typically encountered in dermal applications. In situ formation is novel. The second layer 16 in turn supplies the third layer 18 with moisture. The polymer may also serve the purpose of retaining the liquid to minimize back transport or leakage.

Alternatively or in addition, a chemical gradient can be established for the second layer 16. The second layer 16 media can be precoated with food grade citric acid (or the salt thereof), potassium tartrate, sugar or any other suitable chemical species and dried to produce a chemical gradient. Upon contact with initial fluid from the area of application, the presence of a vast molar excess of citric acid on the distal side of the membrane of the first layer 14 would readily drive aqueous flow into the second layer 16 across the nanofilter until equilibrium was reached on the other side. This serves several key purposes, namely to assure unidirectionality initially, to assure rapid fluid flow, and to assure the quickest assay result. The chemical chosen needs to be compatible with the color chemistry but generally any salt or weak acid would work.

Depending upon size (diameter, thickness), the third layer membrane will need to attract and retain sufficient fluid to drive the assay color chemistry. In some embodiments, the third layer 18 is kept relatively small and discrete in size (e.g., 1-2 mm diameter or as a 0.1×5 mm line) against the backdrop of the second layer 16 (as example, 1 cm diameter) as shown in FIG. 1B, to help reduce assay volume needs. A small size for assay layer 18 also serves to minimize the overall amount of chemical species contained in the patch and serves to focus the read area of the assay to a discrete dot or line. Universal symbols such as + or − can be used for readout. Such symbols can be facilitated through the preprinting of a portion of the symbol, such as a perpendicular negative similar-colored line printed on the outside of layer two 16 or three 18 or four 20. This indicates a default negative result (e.g., sugar-free in the case of beverage testing or non-diabetic normal reading in the case of diabetic screening of mammals). Color development of a perpendicular assay layer 18 line of the same or similar color yields the universally recognized + (positive) result symbol.

The third layer 18 contains the color chemistry. Either direct chemical calorimetric or enzyme based colorimetric chemistries are readily incorporated into the third layer and are well known in the art. Suitable web material would be pre-coated with the appropriate chemistry, cut to appropriate shape, and integrated into the four-layer assembly.

As one example, for direct chemical detection of glucose, a suitable web material is impregnated with a solution and allowed to dry. The solution can include one part copper sulfate, twelve parts sodium hydroxide, four parts sodium carbonate, and fifteen parts citric acid. In this example, a blue color would indicate a negative result and an orange result positive.

As another example, for direct enzymatic detection of glucose, a suitable web material such as GF/B, commercially available from Whatman, cellulose grades 939 or 989, commercially available from Ahlstrom, of Mount Holly Springs, Pa., or FS39, commercially available from Filtration Sciences, of Santa Ana, Calif. are impregnated with 4% glucose oxidase, 0.4% horseradish peroxidase, and 4% orthotolidine as substrate in a suitable buffer and allowed to dry. In some embodiments, the percentages are weight to weight. In other embodiments, the percentages are weight to volume. The intensity of blue color is read against a color key or at a fixed time as a “yes/no” result.

As another example, polyester reinforced polysulfone membrane (commercially available from Pall Specialty Products, of East Hills, N.Y.) is impregnated with a solution containing 3U/mL glucose oxidase, 3 U/ml peroxidase, 3mM amino-antipyrine, 5 mM TOPS in 0.1 M phosphate buffered saline pH 7. The appearance of a violet color and its intensity are directly proportional to glucose concentration. One of ordinary skill in the art will recognize that a unit of enzyme activity ‘U’ is an amount of enzyme required to catalyze conversion of an amount of substrate to product in a defined period of time under conditions of specified pH and temperature.

Some methods for producing glucose oxidase are based on glucose oxidation to gluconate by the action of glucose oxidase enzymes (GOD) and formation of hydrogen peroxide. This step of reaction is specific for glucose. In the next, indicator reaction, the utilization of oxygen is measured or the reaction is based on the hydrogen peroxide formed, which oxidizes a chromogenic substance to a colored product under the action of peroxidase (POD). The intensity of absorbance of thus formed stained product is proportional to the concentration of glucose in the sample. Various chromogenic substances can be used, such as: (i) O-dianizidine; (ii) ABTS (2,2″-azino-di-3-ethylbenzothiazoline sulfonic acid-6-diammonium salt); (iii) 4-aminoantipyrine-dimethylanilline; and (iv) 4-aminoantipyrine

ABTS and 4-aminoantipyrine are most commonly used as a chromogen. Accordingly, there are two methods: (i) GOD-Perid method, and (ii) GOD-PAP method.

In the GOD-Perid method, the specific enzyme glucose oxidase oxidizes glucose with the formation of H202. Thus formed H202 oxidizes ABTS chromogen into a green-stained compound. The absorbance of oxidized, green-stained chromogen is measured photometrically at 436 nm or between 560 and 660 nm. Test strips for quantitative determination of blood glucose concentration are made on the principle of GOD-Perid method. They can be used for fast differentiation of glycemia.

In a GOD-PAP method, the presence of peroxidase, hydrogen peroxide reacts with 4-aminoantipyrine to form a red-stained compound with a maximal absorbance at 510 nm. The 4-aminoantipyrine chromogen is stable and resistant to auto-oxidation, therefore the method is highly reliable and specific for glucose.

By way of reference, the methods described in U.S. Patent Application Publication Nos. 20050003523, 20040126830, 20030170768, and 20030175153 are hereby incorporated by reference in their entireties. Numerous other chemistries are described in the art.

The fourth layer 20, 20′ can be used for visualization of assay results and to retain all the layers of the device 10, 10′ in addition to adhere to the area of placement. The application (beverage or dermal patch) can determine a transparent occlusive material to be used. Occlusive films of polyethylene terephthalate (MYLAR), polyvinylidene chloride, polyvinyl chloride, polyolefin, polystyrene, polyethylene, polypropylene, polyester, polycarbonate, cellulose acetate, PTFE, nitrocellulose, or other material are useable.

A physical gradient involves the use of multiple layers and specific design features to achieve unidirectional flow. Use of a multilayered device for detection of analyte, e.g., glucose, in dispensed soft drink beverages requires that the results be made immediately available to the end-user after dispensing and before consumption. The need for an ultra-rapid result necessitates specific physical design features to facilitate immediate transport of liquid to the test zone in the multilayered assay patch. As such the patch device is typically applied to the outside of a beverage container but that it is understood that that is not the only design means for providing a rapid result. Alternatively fluid is collected by a straw or wick before consumption and applied to the patch on the side of the container or to a device kept altogether separate from the container. The collection and testing device may be a stirrer, paddle, swizzle stick or straw with the color indicator chemistry integrated into it.

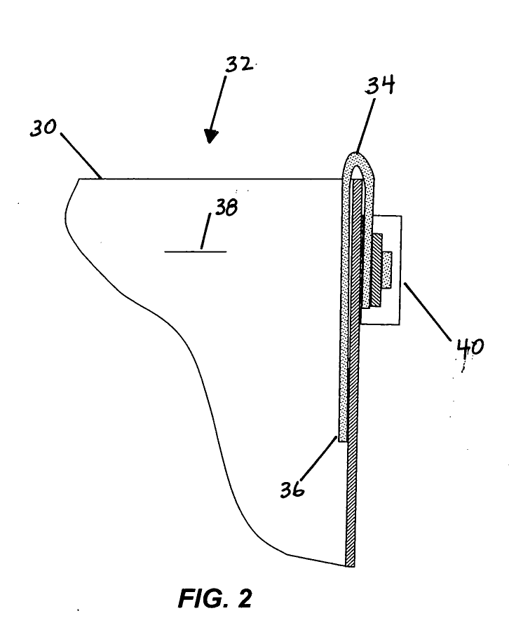

For embodiments in which the device 10 or patch is part of the container, fluid can delivered to the patch assay device by several means. Referring to FIG. 2, fluid can be delivered to the patch over the rim 30 of a container 32 by a wick 34 (such a device is useful for testing hot beverages for caffeine to facilitate sample cooling before testing). One end of an interior portion of the wick 34 extends to below a fluid fill line 30. This ensures that the wick 34 is exposed to fluid when the container 32 is filled. Another end of the wick 34 is in communication with a patch device 40 disposed along an exterior surface. The wick 34 draws a sample of fluid from the container 32 to the patch device 40 for analysis by an one of the means described herein. In some embodiments, the wick can be integrated into the seam of the cup 32 or through an orifice (not shown) in the side of the cup 32 (rather than over the rim 30).

The use of an orifice is enhanced by several means: through the use of gravity to facilitate fill-up of the device outside the container; through liquid level equalization owing to the presence of a hole in the cup at a point lower than in patch layer 4 (this allows for the ‘fill-up’ of liquid in the patch by pressure equalization); through pressure assistance applied by end-user (fluid is forced into the patch through end-user pressing a sealed air bubble to force displacement); through pressure and squeezing of layers together; through creation of a vacuum or negative pressure in the patch on the outside of the container to draw fluid in.

It is understood that a combination of gradients can be used for device construction. The selection and coating of materials, coupled with specific design features can be varied to produce combination gradients. It is possible to construct designs involving combinations of a dryness (moisture) gradient; a hydrophilic gradient for polar water molecules; an absorbent polymer gradient; a chemical equilibrium osmosis gradient; and a physical gradient; or the like.

Suitable water permeable laminating adhesive tapes for multilayer device construction and suitable water impermeable adhesive tapes for patch adhesion are well known in the art. Examples include: ARcare R 8311 and 90374, commercially available from Adhesives Research, Inc., of Glen Rock, Pa.; Dermiclear, commercially available from Johnson and Johnson; and Mn-100, commercially available from Memtec, Minnetonka, Minn.

To facilitate assay read time, a volume indicator means may be added to the multilayered device. Appropriate volume indicator chemistry is added to layer 2. This supplies a contrasting backdrop by which assay layer 3 is read. Suitable materials include pH indicators which when wet turn color, leuco-dyes, food dyes, or silica gel desiccant paper (as layer 2) changing color, from blue to pink to indicate moisture content is adequate.

### EXAMPLE 1

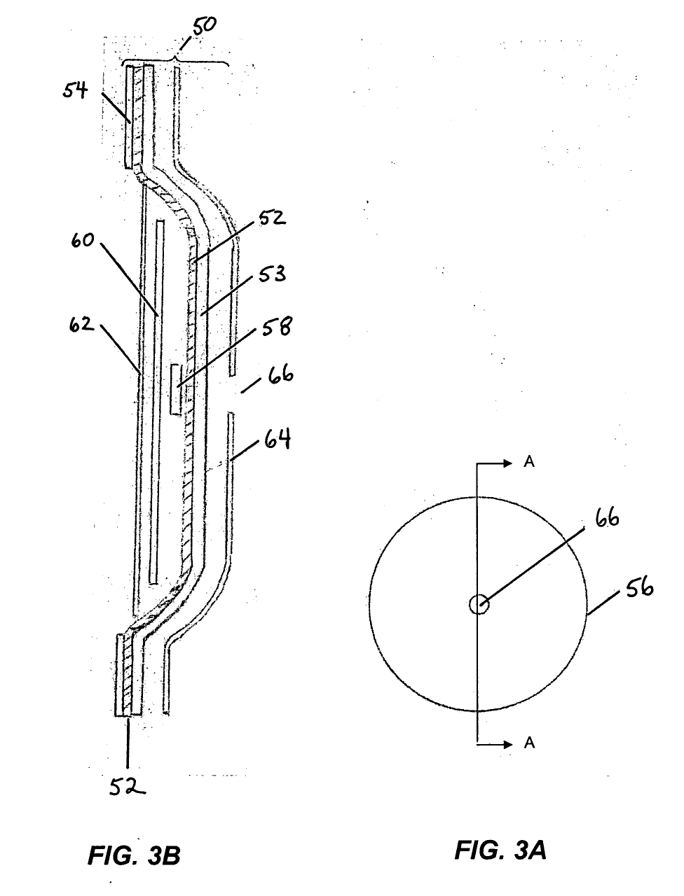

Referring to FIG. 3A and FIG. 3B, a multilayered laminate 50 of acrylic, non-sensitizing, medical grade skin adhesive 50, are adhered to clear 2.0 mil thick occlusive layer 53 and 60# siliconized Kraft release liner paper 54. Each of the layers can be dye cut into a preferred size and shape, such as 2.5 cm diameter circles 56. A smaller, 1.5 cm circle of release line is removed from the center of the triple-layered laminate (not shown) exposing the underlying adhesive and leaving a 0.5 cm width peripheral ring of release paper intact 54. Suitable skin adhesives include HY-3 High MVTR, commercially available from Adhesives Research, Inc., Glen Rock, Pa. The occlusive layer 53 can be formed from polyethylene terephthalate (PET), commercially available from Polyester Converters, of Fullerton, Calif.

A method described in Trinder, P., Ann. Clin. Biochem. 6 (1969) 24, can be modified for colorimetric detection of glucose. Sheets of 0.45 micron polyester reinforced polysulfone membrane 58 (commercially available from Pall Specialty Products, of East Hills, N.Y.) can be impregnated by immersion in a coating solution containing 3U/mL glucose oxidase, 3 U/ml peroxidase, 3 mM amino-antipyrine, 5 mM TOPS in 0.1 M phosphate buffered saline pH 7.0 and allowed to air dry. Suitable chemicals are commercially available from Sigma-Aldrich, of St. Louis, Mo. Two and a half mm diameter circles of assay reagent coated membrane 58 can also be dye cut and adhered to the center of the laminate prepared above.

Next a 1.0 cm diameter disk of dry, hydrophilic GF/B membrane 60 (commercially available from Whatman) can be adhered centrally to the laminate fully covering the assay reagent layer. On top of that is adhered a 1.5 cm diameter disk of 20 nm pore size ion track-etched polycarbonate membrane 62 (commercially available from SPI).

The four-layered stack 50 can be placed on the surface of a beverage cup half way up the side by removing the release liner 54. The center region of the patch 50 is placed over a 1.5 mm diameter hole (not shown) in the side of the cup. On top of the transparent patch, an opaque label 64 can be attached with a visual read hole 66 centered over the reagent layer 53.

In a test of the device, patch containers were filled blind to the top of the cup, in triplicate, with three different types of sugar-free beverage and three different types of sugar containing beverage. Results, read at 1 minute at the observation point 66 were as listed in Table 1.

TABLE 1
Test Results

| Beverage Type | Color Result |
|---|---|
| Sugar-free Brand 1 | Neg (all 3 replicates) |
| Sugar-free Brand 2 | Neg (all 3 replicates) |
| Sugar-free Brand 3 | Neg (all 3 replicates) |
| Sugar-containing Brand 1 | Pos (all 3 replicates) |
| Sugar-containing Brand 2 | Pos (all 3 replicates) |
| Sugar-containing Brand 3 | Pos (all 3 replicates |

### EXAMPLE 2

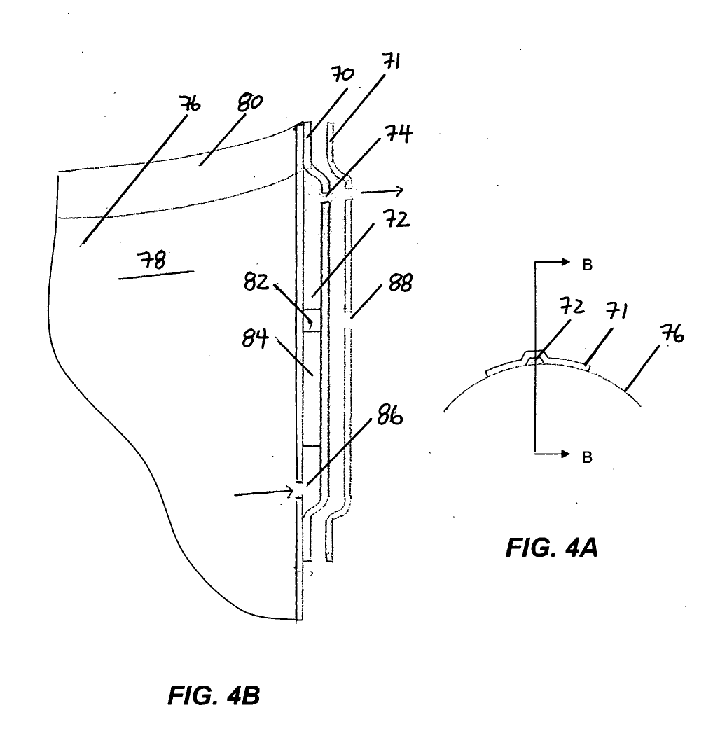

Referring to FIG. 4A and FIG. 4B, a laminate of acrylic medical grade adhesive HY-3 High MVTR (commercially available form Adhesives Research, Inc., of Glen Rock, Pa.) is adhered to clear 2.0-mil thick polyethylene terephthalate (PET) (commercially available Polyester Converters, of Fullerton, Calif.) (shown herein as 70 and 60# siliconized Kraft release liner paper (not shown) can be cut into a 1.0 cm wide×7 cm long strip. A 1.0 mm wide×6.5 cm long×0.25 mm deep channel 72 can be thermoformed into the trilayer laminate by pressing the laminate into a heated aluminum mold containing a raised aluminum line of comparable size on one side and a corresponding depression of comparable size on the other. The release liner side of the laminate can be placed toward the raised side in order to create a microchannel 72 in the laminate surface raised away from the point of application.

Next a 0.15 mm air pinhole 74 is made in the laminate channel 72 at the top end, at the 6.0 cm position, 1.0 cm from the top side of the strip applied to the surface of the container 76. This hole 74 provides a vent allowing air to exit from the microchannel 72. The hole is located above a container fluid fill line 78 but below the container top 80. The pinhole 74 allows for the gravity driven equalization of pressure in both the container and in the micro-channel 72 to physically assure a rapid transport of fluid past a color indicator layer 82 contained within the micro-channel 72, below the vent 74, and well below the fill line 78 of the container. This configuration provides a fluid level equalization gradient using physical means. Below the pinhole 74 and below the fluid fill line 78 for the container, a 1.0 mm wide×0.25 cm long piece of the reagent layer 82 as described in the first example, was placed in the channel 72 at the 3.5 cm position measured from the top of the strip and adhered to the channel surface after removal of the release liner from the thermo-molded patch. Immediately below that a 1.0 mm wide×1.0 cm long piece of GF/B 84 (commercially available from Whatman) was placed in the micro-channel 72 adjacent and directly below the reagent layer 82 in direct lateral flow contact with it.

The device 71 can be adhered to the side of a beverage container 76 with the top end of the strip 71 and vent hole 74 directly below the container top lip 80. At the bottom position of the channel 72 at a point measured 1.25 cm from the bottom of the strip 71, a 0.35 mm diameter fluid inlet hole 86 can be made in the container 76 wall but not in the laminate itself. The size of this fluid hole 86 is preferably significantly larger than the air escape hole 74 to assure rapid unidirectional flow of fluid to the detection means. As such, the microchannel 72 in the patch device 71 on the container 76 has a 0.35 mm fluid inlet hole 86 at one end of the macro fluidic channel (in the container wall) and a 0.15 air escape hole 74 in the MYLAR wall at the other end of the device 71, just below the rim 80 of the container 76.

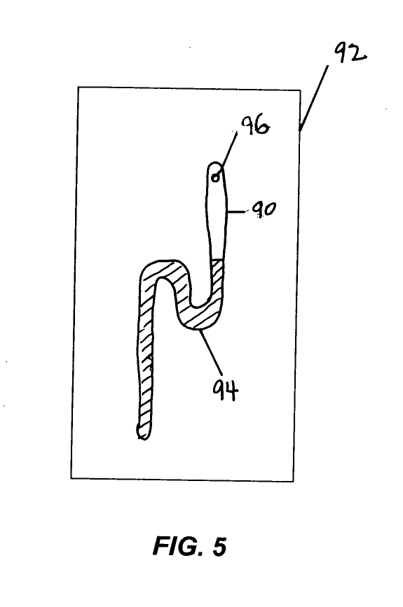

To run the assay, the container is filled to the fill line 78. Pressure between the fluid in the cup 76 and in the micro-channel 72 forced the fluid past the reagent zone hence enabling rapid color development. Violet color (positive for sugar) appears as a 1.0 mm wide×0.25 cm long visually dissemble line 88 after about 30 seconds on the outside of the container 76. Testing using the same beverage solutions as in Example 1, above yielded the same results. To prevent any back flow into the beverage container, the above design can be modified. For example, as shown in FIG. 5, a microchannel 90 can be thermoformed into a device 92 forming a hairpin, or S-shaped loop, to create a simple backflow valve 94 as found in a sink drain. This feature eliminated back flow observed in earlier prototypes and also yielded comparable results. A vent 96 is visible at a top end of the microchannel 90.

The present invention may be embodied in other specific forms without departing from the spirit or essential characteristics thereof. The presently disclosed embodiment is therefore to be considered in all respects as illustrative and not restrictive, the scope of the invention being indicated by the appended claims, rather than the foregoing description, and all changes which come within the meaning and range of equivalency of the claims are, therefore, to be embraced therein. All references cited are hereby incorporated herein by reference in their entirety.

What is claimed is:

1. An integral fluid collection device for collecting a fluid sample from a sample surface comprising:
at least three internal layers of which a first layer includes a hydrophilic nanofiltration material adhered to a second layer;
the second layer includes absorbent material adhered to a third layer;
the third layer includes an assay detection zone having integrated direct chemical calorimetric or enzymatic-based calorimetric detection suitable for detecting an analyte, the third layer adhered to a fourth layer; and
a fourth layer including a visually-clear occlusive material, and being external to the first, second and third layers, the fourth layer also having an area greater than the at least three internal layers so as to overlap the three internal layers at an area of contact with the sample surface.

2. The apparatus of claim 1, wherein the fourth layer comprises an adhesive zone peripheral to the three internal layers.

3. The apparatus of claim 2, wherein non-aqueous permeable double-stick adhesive layer is provided along the adhesive zone to promote containment of fluid within the device, when adhered to the sample surface.

4. The apparatus of claim 3, wherein the adhesive layer is covered by a releasable liner.

5. The apparatus of claim 1, wherein the hydrophilic nanofiltration material comprises a membrane defining substantially straight-through pores acting as a molecular sieve to allow for immediate passage of fluid.

6. The apparatus of claim 5, wherein the maximum pore diameter is not more than about 200 nm.

7. The apparatus of claim 1, further comprising a gradient from the area of contact with the sample surface to the assay detection zone, the gradient selected from the group consisting of: dryness (moisture) gradient; a hydrophilic gradient for polar water molecules; an absorbent polymer gradient; a chemical equilibrium osmosis gradient; a physical gradient; and combinations thereof.

8. A method for detecting a target analyte obtained from a sample surface comprising:
obtaining a patch comprising: at least three internal layers, the first layer having hydrophilic nanofiltration material adhered to a second layer; the second layer having absorbent material adhered to a third layer; the third layer comprising an assay detection zone having integrated direct chemical colorimetric or enzymatic-based colorimetric detection suitable for detecting an analyte, the third layer adhered to a fourth layer; the fourth layer having a visually-clear occlusive region and being external to the first, second, and third layers and having an area greater than the three internal layers so as to overlap the three internal layers;
applying the patch to the sample surface; and
viewing through the visually-clear occlusive region a calorimetric change in the patch, wherein the colorimetric change indicates the presence of the target analyte.

9. The method of claim 8, further comprising collecting the sample from the sample surface.

10. The method of claim 8, wherein the sample surface comprises a mammalian skin.

11. The method of claim 8, wherein the sample surface comprises a fluid vessel.

12. The method of claim 8, further comprising establishing a gradient from the sample surface to the assay detection zone, the gradient selected from the group consisting of: dryness (moisture) gradient; a hydrophilic gradient for polar water molecules; an absorbent polymer gradient; a chemical equilibrium osmosis gradient; a physical gradient; and combinations thereof.

13. The method of claim 8, wherein the fourth layer has a release liner and an adhesive zone peripheral to the three internal layers.

14. The method of claim 8, further comprising forcing a fluid sample into the patch through application of end-user pressure.

15. A method for producing an analyte detection patch for collecting a sample from a sample surface comprising:
providing a first layer having hydrophilic nanofiltration material;
providing a second layer having absorbent material;
providing a third layer comprising an assay detection capability including integrated direct chemical calorimetric or enzymatic-based colorimetric detection chemistry suitable for detecting an analyte; and
providing a fourth layer comprising a visually-clear occlusive material, the fourth layer being external to the first, second and third layers and having an area greater than these three internal layers, the fourth layer also having a release liner and an adhesive zone peripheral to the three internal layers.

16. The method of claim 15, further comprising adhering the first layer to the second layer, adhering the third layer to the first and second layers and overlapping the three internal layers with the fourth layer.

17. The method of claim 15, further comprising establishing a gradient from the sample surface to the assay detection zone, the gradient selected from the group consisting of: dryness (moisture) gradient; a hydrophilic gradient for polar water molecules; an absorbent polymer gradient; a chemical equilibrium osmosis gradient; a physical gradient; and combinations thereof.

18. The method of claim 15, wherein the hydrophilic nanofiltration material comprises a membrane defining substantially straight-through pores acting as a molecular sieve to allow for immediate passage of fluid.

19. The method of claim 15, wherein the absorbent material comprises at least one of a cellulosic material and a glass fiber material.

20. The method of claim 15, further comprising defining a narrow channel within the fourth layer, the narrow channel containing the first, second, and third layers.
